(19) United States
(12) Patent Application Publication
(10) Pub. No.: US 2025/0231174 A1
(43) Pub. Date: Jul. 17, 2025

(54) METHODS AND DEVICES FOR SCREENING EXTRACELLULAR VESICLES

(71) Applicant: THE REGENTS OF THE UNIVERSITY OF MICHIGAN, Ann Arbor, MI (US)

(72) Inventors: Zeqi Niu, Ann Arbor, MI (US); Sunitha Nagrath, Ann Arbor, MI (US); Yoon-Tae Kang, Ann Arbor, MI (US)

(21) Appl. No.: 18/854,390

(22) PCT Filed: Apr. 7, 2023
(86) PCT No.: PCT/US2023/017872

§ 371 Date: Oct. 4, 2024

Related U.S. Application Data
(60) Provisional application No. 63/328,464, filed on Apr. 7, 2022.

(51) Int. Cl. G01N 33/50; B01L 3/00; G01N 33/543

(52) U.S. Cl. CPC G01N 33/5005; B01L 3/502715; G01N 33/54306; B01L 2200/027; B01L 2300/0861

(57) ABSTRACT

The present disclosure provides methods, devices, and kits useful for screening or characterizing extracellular vesicles (e.g., exosomes) by monitoring cell-type specific extracellular vesicle uptake and/or cells' feedback responses.

### CROSS-REFERENCE TO RELATED APPLICATIONS

This application claims the benefit of U.S. Provisional Application No. 63/328,464, filed Apr. 7, 2022, the content of which is herein incorporated by reference in its entirety.

### FIELD

The present disclosure provides method and devices for screening or characterizing extracellular vesicles (e.g., exosomes) utilizing cell-type specific extracellular vesicle uptake and cells' feedback responses.

### BACKGROUND

Analysis of extracellular vesicles (EVs) is becoming an increasingly promising diagnostic field, particularly for effective biomarker for cancers. The primary function of EVs is to facilitate cell-cell communication, and information from their progenitor cells makes them the ideal biomarkers for early cancer diagnoses and metastasis. The presence of EVs in various biological fluids secreted by the body can be used for isolating and analyzing exosomes for cancer diagnoses in the form of liquid biopsy. However, methods for effective separation of cancer-specific exosomes are limited due to volume requirements, time, sample complexity, heterogeneity amongst exosomes, and overlap of physical and chemical properties with other EVs contained in a sample. Additionally, current downstream analysis methods lack the ability to differentiate exosomes of different origins in a sample.

### SUMMARY

Disclosed herein are methods for characterizing or screening extracellular vesicles in vitro. In some embodiments, the methods comprise incubating a sample comprising extracellular vesicles with one or more cell types; measuring uptake of the extracellular vesicles in each of the one or more cell types; and determining cellular origin of the extracellular vesicles. In some embodiments, the methods further comprise removing resulting sample following incubation.

In some embodiments, the cellular origin of the extracellular vesicles in the sample is that origin of the one or more cell types having increased uptake levels of the extracellular vesicles in the sample when compared to control uptake levels of the one or more cell types for extracellular vesicles of a different origin.

In some embodiments, the one or more cell types comprises at least one organ- or tissue-specific cell type. In some embodiments, the one or more cell types comprises two or more cell types. In some embodiments, each of the two or more cell types are organ- or tissue-specific cell types.

In some embodiments, measuring the uptake comprises comparing uptake between each of the two or more cell types and calculating a relative uptake value for each of the two or more cell types. In some embodiments, the origin of extracellular vesicles is determined as the cell type having highest relative uptake value from the two or more cell types.

In some embodiments, the one or more cell types comprises cancer cell types.

In some embodiments, the one or more cell types are immobilized on a solid support. In some embodiments, each of the one or more cell types are immobilized in discrete locations on the solid support. In some embodiments, the solid support comprises a microfluidic device.

In some embodiments, the extracellular vesicles comprise exosomes. In some embodiments, the extracellular vesicles comprise cancer cell-derived exosomes.

In some embodiments, the methods further comprise labeling the extracellular vesicles with a detectable marker. In some embodiments, the labeling is prior to incubating the sample with one or more cell types. In some embodiments, the labeling follows incubating the sample with one or more cell types.

In some embodiments, the detectable marker comprises a fluorophore. In some embodiments, the detectable marker comprises an extracellular vesicle binding agent. In some embodiments, the extracellular vesicle binding agent comprises a tetraspanin protein (e.g., CD63, CD8, CD91), annexin V, or an antibody (e.g., EGFR, EpCAM).

In some embodiments, the methods further comprise measuring extracellular vesicle secretion from the one or more cell types. In some embodiments, determining the origin of extracellular vesicles is further determined by the cell type having a minimal amount of vesicle secretion.

In some embodiments, the methods further comprise measuring one or more biomarkers in the sample. In some embodiments, the one or more biomarkers comprise a cancer biomarker. In some embodiments, the cancer biomarker is circulating tumor DNA.

In some embodiments, the sample is a biological sample. In some embodiments, the biological sample is obtained from a subject. In some embodiments, the subject has or is suspected of having a disease or disorder. In some embodiments, the subject had or is expected to have a medical procedure, treatment, or intervention. In some embodiments, the methods comprise comparing cellular origin of the extracellular vesicles from the subject before and after the medical procedure, treatment, or intervention. In some embodiments, the methods further comprise diagnosing or prognosing a disease or disorder in the subject.

Further disclosed herein is a microfluidic device. The microfluidic device comprises one or more incubation chambers, each chamber comprising cells of a single type tethered to a surface of the chamber. In some embodiments, the one or more incubation chambers are in fluid communication with each other. In some embodiments, the methods are carried out using a microfluidic device as disclosed herein.

Further disclosed herein is a system comprising the microfluidic device disclosed herein and a sample comprising extracellular vesicles. In some embodiments, the sample is in the one or more incubation chambers.

Additionally disclosed herein are kits comprising a microfluidic device as disclosed herein or components necessary for fabricating a microfluidic device as disclosed herein.

Other aspects and embodiments of the disclosure will be apparent in light of the following detailed description and accompanying figures.

### BRIEF DESCRIPTION OF THE DRAWINGS

FIG. 1 is a schematic of an exemplary ^{Cell}ExoChip microfluidic device conjugated with live cancer cells for evaluating specific exosome uptake by their mother cells to study exosomes.

FIG. 2A is a schematic of cancer cell biotinylation for on-chip conjugation. FIG. 2B is an image of an exemplary ^{Cell}ExoChip showing relative dimensions. FIG. 2C is a graph of on-chip cell deposition rate with/without biotinylation on avidin-conjugated device surface. FIG. 2D is a scanning electron microscope image of live cancer cell immobilization on chip via avidin-biotin chemistry

FIGS. 3A-3D show viable cancer cell immobilization cell-exosome specific uptake. FIG. 3A is a graph of on-chip cell deposition rate with/without biotinylation. FIG. 3B is images of the viability of cells on chip with live (left) and dead (right) dye staining. FIG. 3C is a graph of the specific uptake of lung cancer exosomes by three different cells from different organs. FIG. 3D are imagines of the uptake of lung cancer exosomes by two different lung cancer cells on chip.

FIGS. 4A-4D is a comparison study of cellular uptake of exosomes from three different organs. FIG. 4A is a schematic diagram of specific cell-exosome uptake evaluation study. FIGS. 4B and 4C are fluorescence images of cellular uptake at lung cancer cells with lung exosomes (FIG. 4B) and breast cancer cells with lung exosomes (FIG. 4C). FIG. 4D are graphs of the relative EV uptake at different cell-exo combinations.

FIGS. 5A-5D show exosome secretion from live cells on ^{Cell}ExoChip with/without

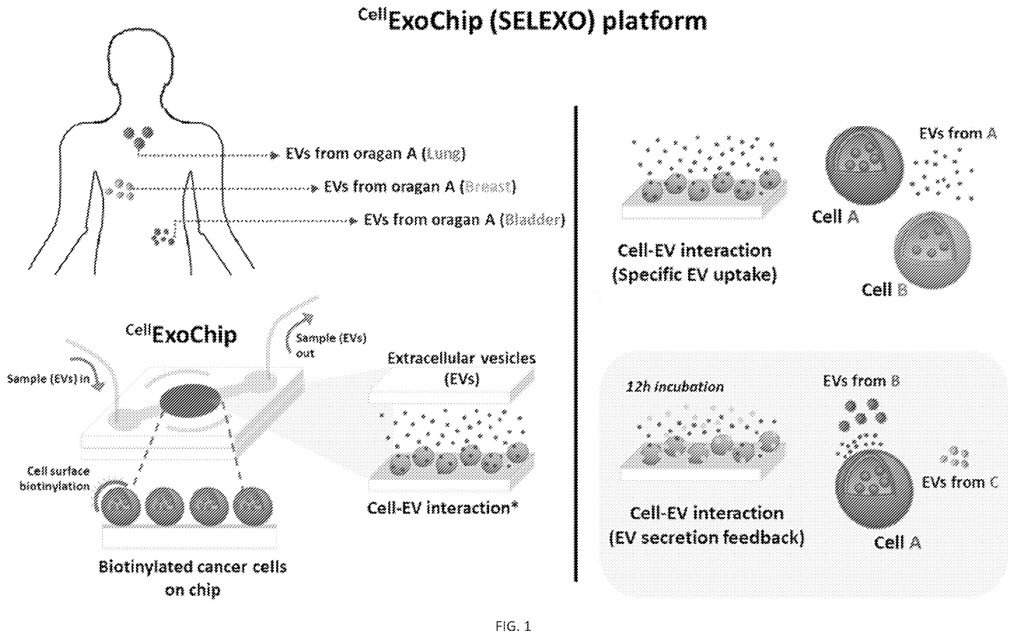

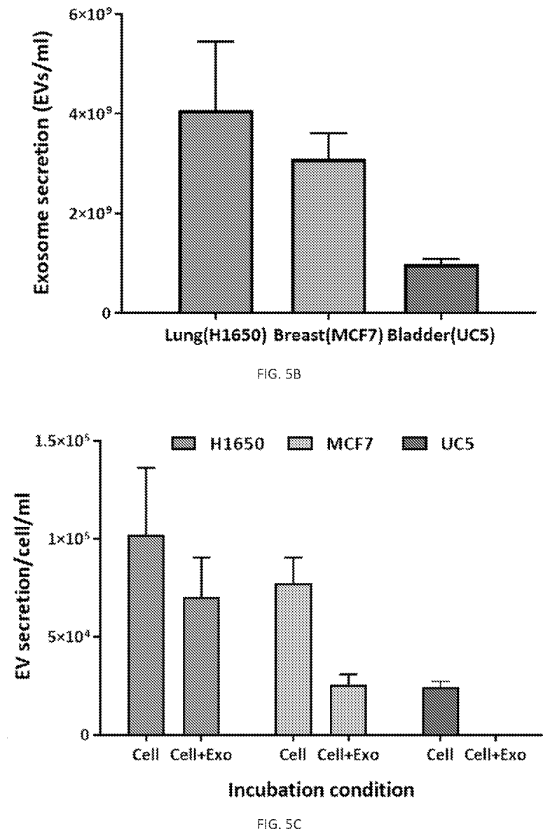

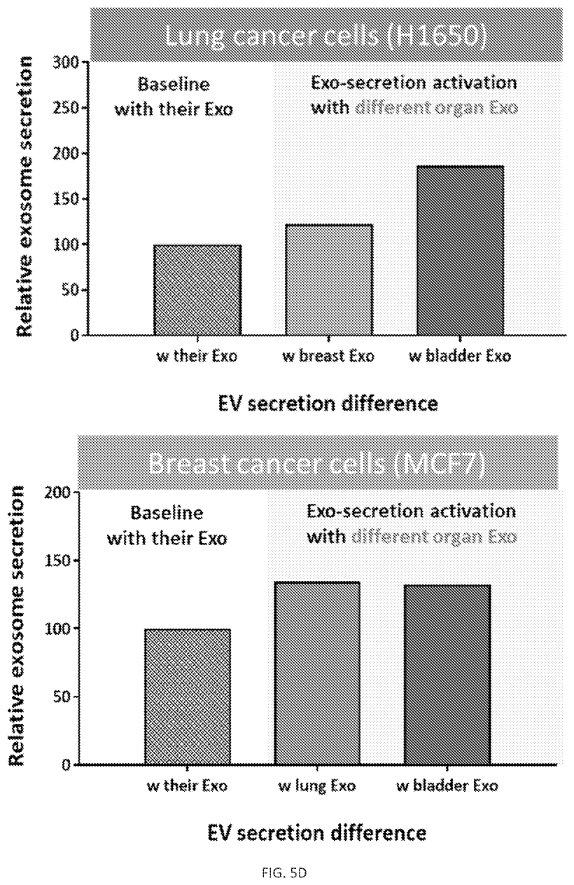

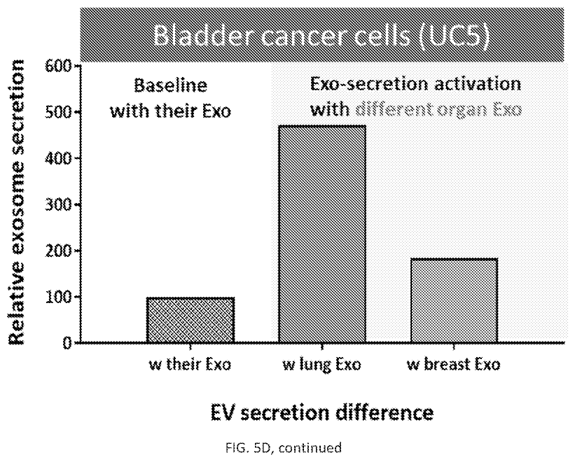

extrinsic exosome introduction. FIG. 5A is a scanning electroscope image of exosomes secreted by cells on chip. FIG. 5B is a graph of the exosome secretion rate comparison between three different cells without exosome introduction. FIG. 5C is a graph of the exosome secretion rate change before/after their exosome introduction to the chip. FIG. 5D is graphs of the relative exosome secretion change after introduction of exosomes from different organs.

### DETAILED DESCRIPTION

Disclosed herein are methods and devices for the characterization and screening of extracellular vesicles (EVs), for example exosomes, e.g., cancer-derived exosomes. The methods bypass the constraint of analysing and profiling embedded proteins prior to EV isolation.

The microfluidic device facilitates an interaction between cells and EVs, for example from a liquid biopsy, due to the presence of certain proteins in EVs, enabling preferential uptake of the exosomes by organ-specific cells, due to so-called organotropism. As described herein, the uptake of lung cancer cell exosomes into three different cancer cell lines (lung, bladder, and breast) was measured on the device and the relative uptake of lung cell exosomes by the respective lung cells was 100% compared to the bladder cells and breast cell which were 15.87% and 40.31%, respectively.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

A “biomarker” includes a biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, a cell fragment, and a small molecule. that is present in a biological sample and that may be isolated from, or measured in, the biological sample. Furthermore, a biomarker may be the entire intact molecule, or a portion thereof that may be partially functional or recognized, for example, by an antibody or other specific binding protein. A biomarker may be associated with a given state of a subject, such as a particular stage of disease. In some embodiments, the biomarker is a cancer biomarker (e.g., circulating tumor DNA, protein biomarkers (e.g., prostate specific antigen, alpha-fetoprotein, carcinoembryonic antigen). A measurable aspect of a biomarker may include, for example, the presence, absence, or concentration of the biomarker in the biological sample from the subject and/or relative changes of any of the measurable aspects compared to a standard (e.g., internal or from a healthy subject). The measurable aspect may also be a ratio of two or more measurable aspects of two or more biomarkers. Biomarker, as used herein, also encompasses a biomarker profile comprising measurable aspects of two or more individual biomarkers. The two or more individual biomarkers may be from the same or different classes of biomarkers such as, for example, a nucleic acid and a carbohydrate, or may measure the same or different measurable aspect such as, for example, absence of one biomarker and concentration of another. A biomarker profile may comprise any number of individual biomarkers or features thereof. In another embodiment, the biomarker profile comprises at least one measurable aspect of at least one internal standard. Methods of identifying and quantifying biomarkers are well known in the art and include histological and molecular methods such as enzyme-linked immunosorbent assays (ELISA) and other immunoassays, gel electrophoresis protein and DNA arrays, mass spectrometry, colorimetric assays, electrochemical assays, and fluorescence methods.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. CHARACTERIZING EXTRACELLULAR VESICLES

The present disclosure provides methods for characterizing extracellular vesicles. The methods may comprise incubating a sample comprising extracellular vesicles with one or more cell types; measuring uptake of the extracellular vesicles in each of the one or more cell types; and determining origin of the extracellular vesicles in the sample.

The origin of the extracellular vesicles can be identified by which cell-types show preferential uptake of the extracellular vesicles. In some embodiments, the cellular origin of the extracellular vesicles in the sample is that origin of the one or more cell types having increased uptake levels of the extracellular vesicles in the sample when compared to control uptake levels of the one or more cell types for extracellular vesicles of a different origin.

In some embodiments, measuring the uptake comprises comparing uptake between each of two or more cell types and calculating a relative uptake for each of the two or more cell types. In some embodiments, the origin of extracellular vesicles is determined by the cell type having the highest relative uptake of the extracellular vesicles.

The methods may further comprise measuring extracellular vesicle secretion from the one or more cell types. In some embodiments, vesicle secretion is measured during incubation with the one or more cell types. In some embodiments, vesicle secretion is measured following incubation with the one or more cell types. When the origin of the extracellular vesicles is the same as that of the cell type with which it is incubated, the cells secrete fewer new extracellular vesicles. However, when the origin of the extracellular vesicles is different to that of the cell type with which it is incubated, the cells secrete increased amounts of new extracellular vesicles. As such, in some embodiments, determining the origin of extracellular vesicles is further comprises identifying the cell type having a minimal amount of vesicle secretion.

### a) Incubation

The methods comprise incubating a sample comprising extracellular vesicles with one or more cell types. The incubation times can vary and are in no way limiting. For example, incubation can be anywhere between 10 minutes to overnight (e.g., 16 hours). The incubation can be with or without agitation, and the agitation during the incubation period can be constant or intermittent. The incubation can be at any temperature to support cell viability, generally, at or about 37° C. In some embodiments, the incubating further comprises using buffer or media components (e.g., tissue extracts, and the like) to support cell viability. The incubation may further comprise providing cells an environment in which to maintain viability. For example, during the incubation nutrients, gases (oxygen or CO₂, etc.), chemicals, or proteinaceous/non-proteinaceous stimulants may be provided to the cells.

Following the incubation, the resulting sample can be removed from the cells. Once the resulting sample is removed, the cells may be washed with an appropriate buffer or solution that removes non-specific interactions of extracellular vesicles.

The disclosed methods are not limited by cell type. Examples of cell types that may be included in the cell models of the invention include, without limitation, human cells, animal cells, mammalian cells, and/or diseased cells (e.g., cancer cells, bacterially or virally infected cells). In some embodiments, the one or more cell types comprises at least one organ- or tissue-specific cell type. For example, the cell type may be derived from a certain organ or tissue (e.g., lung, brain, breast, etc.) or the cell type may a precursor or progenitor cell to the desired organ or tissue. In some embodiments, the one or more cell types comprises two or more (e.g., three, four, five, six, seven, eight, nine, ten or more) cell types. In some embodiments, each of the cell types are organ- or tissue-specific cell types. As such, each of the cell types may be specific to a different organ or tissue. Alternatively, some or all of the one or more cell types may be to the same organ or tissue.

In some embodiments, the one or more cell types comprises cancer cell types. In certain embodies, one or more of the cell types are cancer cell types. In certain embodiments, each of the cells types are cancer cell types. The cancer cell types may be cancer cells derived from different organs or tissues. The cancer cell types may be cancer cells derived from different types of cancer (e.g., sarcoma, carcinoma, lymphoma). The cancer cells types may be all the same type (e.g., all breast cancer cells), but may be different established cell lines. Examples of cancer cell types include, without limitation, lung cancer, breast cancer, prostate cancer, melanoma, squamous cell carcinoma, colorectal cancer, pancreatic cancer, thyroid cancer, endometrial cancer, bladder cancer, kidney cancer, solid tumor, leukemia, and non-Hodgkin lymphoma. Numerous cancer cell lines are known in the art, and any such cancer cell line may be useful in the methods disclosed herein.

In some embodiments, the cells of the one or more cell types are immobilized on a solid support. “Solid support,” as used herein. refers any solid device or structure capable to immobilize cells on a surface. For example, the solid support may be an array with a spatially defined areas in which individual cells or cell types are isolated by a surface treatment or affinity methods (surface modifications or ligand binding). The solid support may have distinct structures (e.g., chambers, sections, wells, or channels) which separate the individual cells or cell types, for example, a microfluidic device or a microtiter plate. In some embodiments, the methods further include isolating each of the one or more cell types into individual locations within a solid support.

The solid support may be composed of any of a wide variety of materials, for example, polymers, plastics, resins, polysaccharides, silica or silica-based materials, carbon, metals, inorganic glasses, membranes, or any combinations thereof. The solid support material may be treated, coated, modified, printed or derivatized using polymers or chemicals to impart desired properties or functionalities to the support surface. Preferred solid support material may be compatible with the range of conditions encountered during the disclosed methods including salt concentrations, pHs, temperatures, and be optically transparent.

The solid support may be smooth, having a substantially planar surface, or it may contain a variety of structures such as wells, grooves, depressions, channels, elevations, chambers, or the like, in which individual cells or cell types are isolated. The solid support may be a microfluidic device comprising a series of microchannels or chambers which isolate individual cells or cell types within the microchannels or within defined incubation chambers. The solid support may be a multi-well plate comprising a vast number of wells which isolate the individual cells or cell types. The solid support may be a particle or bead (e.g., a nanoparticle or microparticle). In some embodiments, the solid support is a microfluidic device.

In some embodiments, the solid support is functionalized to facilitate immobilization of the cells to the surface of the solid support (e.g., grooves, depressions, channels, elevations, chambers, or the like). Tethering of the cells to the surface may be accomplished by numerous methods known in the art, including, for example, avidin-streptavidin, biotin, and/or the use of a linker. In some embodiments, the solid support is functionalized with a binding agent to facilitate immobilization of the cells to the surface of the solid support. The binding agent can also be directly bound to the solid support using coupling agents such as bifunctional reagents or can be indirectly bound. In some embodiments, the cells are biotinylated and the surface of the solid support is avidin-conjugated, such that the tethering is due to a biotin-avidin association.

The present disclosure further provides a microfluidic device for use in characterizing extracellular vesicles. The microfluidic device may comprise one or more incubation regions (e.g., chambers). In some embodiments, each chamber comprises cells of a single type (e.g., organ- or tissue specific, disease cells) tethered to the surface of the chamber.

In some embodiments, the one or more incubation chambers are in fluid communication with each other. For example, the device may comprise a single sample inlet for loading a single aliquot of sample in each of the incubation chambers. Alternatively, the fluid communication may be controllable such that a resulting sample following an incubation in a first chamber is loaded in a second chamber for a subsequent incubation. In some embodiments, each of the chambers are independent from each other. For example, each chamber has an independent sample inlet and outlet. As such, the sample is divided into aliquots prior to loading into each of the individual chambers.

The disclosed microfluidic devices are suitable for use in the methods characterizing extracellular vesicles as described herein.

### b) Sample

As used herein, the term “sample” is used in its broadest sense. In one sense, it is meant to include a specimen obtained from any source, including biological and environmental samples. Biological samples may be obtained from animals (including humans) and encompass fluids, solids, and/or tissues. Such examples are not however to be construed as limiting the sample types. In some embodiments, the sample is a fluid sample such as a liquid sample. Examples of liquid samples suitable for use with the devices disclosed herein include bodily fluids (e.g., blood, serum, plasma, saliva, urine, ocular fluid, semen, sputum, sweat, tears, and spinal fluid), samples from supernatants and excreted materials of in vitro cultured tissues or cells, water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. Liquid samples can be made from solid, semisolid, or highly viscous materials, such as fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation. Samples can comprise biological materials, such as cells, microbes, organelles, and biochemical complexes. In some embodiments, the samples are cell-free, microbe-free, and/or organelle-free.

The biological sample may be obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. In some embodiments, the sample is blood or blood products. Blood products are any therapeutic substance prepared from human blood. This includes whole blood; blood components (e.g., red blood cell concentrates or suspensions; platelets produced from whole blood or via apheresis; plasma; serum and cryoprecipitate); and plasma derivatives (e.g., coagulation factor concentrates).

In some embodiments, the sample is a biological sample obtained from a subject having or suspected of having a disease or disorder. In some embodiments, the sample is a biological sample obtained from a subject having or suspected of having cancer. In some embodiments, the sample is a biological sample obtained from a subject having or suspected of having a neurodegenerative disease. In some embodiments, samples are obtained from a subject throughout the course of a disease or disorder or during treatment for a disease or disorder and the samples are analyzed for changes in the characterization of the extracellular vesicles over the time period of sample collection.

The term “cancer” refers to a class of diseases characterized by development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The disclosed methods may be useful to wide range of cancer samples including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus.

Neurodegenerative diseases or disorders refer to a group of diseases and disorders that affect the structure or function of the nervous system (e.g., brain and/or spinal cord). Neurodegenerative diseases occur as a result of neurodegenerative processes, e.g., the progressive loss of structure or function of neurons, including but not limited to the death of neurons. Exemplary neurodegenerative diseases include amyotrophic lateral sclerosis, Parkinson's disease, Alzheimer's disease, Huntington's disease, Batten disease, dementia with Lewy bodies, frontotemporal dementia, cerebrovascular dementia, multiple sclerosis, Alexander's disease, Alper's disease, ataxias (e.g., ataxia telangiectasia, Friedrich's ataxia), Canavan disease, corticobasal degeneration, Creutzfeldt-Jakob disease, Gerstmann-Sträussler-Scheinker syndrome, multiple system atrophy, narcolepsy, spinocerebellar ataxia, and the like.

In some embodiments, the sample is a biological sample obtained from a subject who had or is having a medical procedure, treatment, or intervention. In some embodiments, a sample obtained from a subject before and after a medical procedure, treatment, or intervention and both are analyzed for changes in the characterization of the extracellular vesicles. Thus, the methods herein may be used to follow the success of the medical procedure, treatment, or intervention. The type of medical procedure, treatment, or intervention is not limiting to the methods described herein. Exemplary medical procedures, treatments or interventions include: surgeries and surgical excisions or resections, transplants, biopsies, full or focal ablations (e.g., of a tumor or a tissue), administration of an active agent (e.g., pharmaceutical or therapeutic agent), insertion of a fiducial, brachytherapy seed, or drug depot, placement of devices (e.g., stents, grafts, implants, prosthetics), radiation therapy, and the like.

The sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like. In some embodiments, the sample is enriched for extracellular vesicles.

In some embodiments, measuring the uptake and cellular feedback response comprises detection of fluorescence in the one or more cell types.

The methods are suitable for any volume of sample.

### c) Extracellular Vesicles

A unified vesicle nomenclature and classification system utilizing broadly accepted definitions has been elusive in the field. The term “extracellular vesicles,” as used herein, refers to a lipid membrane particles having a diameter (or largest dimension where the particles is not spheroid) of between about 30 nm and 10,000 nm. Extracellular vesicles encompass exosomes, ectosomes, microvesicles, microparticles, prostasomes, tolerosomes (which induce immunological tolerance to dietary antigens), apoptotic bodies (released by apoptotic cells), and nanovesicles. The term “exosome,” as used herein, refers to a membranous particle having a diameter (or largest dimension where the particles is not spheroid) of between about 30 nm and 150 nm, wherein at least part of the membrane of the exosomes is directly obtained or derived from a cell. Most commonly, exosomes will have a size (average diameter) that is up to 5% of the size of the donor cell. Therefore, especially contemplated exosomes include those that are shed from a cell. As used herein, it is not intended that an extracellular vesicle or exosome of the invention be limited by any particular size or size range.

In some embodiments, the extracellular vesicles comprise exosomes. Exosomes may include any shed membrane bound particle that is derived from either the plasma membrane or an internal membrane. Exosomes can also include cell-derived structures bounded by a lipid bilayer membrane arising from both herniated evagination separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen including tumor-derived microRNAs or intracellular proteins. Exosomes can also include membrane fragments.

Exosomes and extracellular vesicles may be released by mammalian cells for a number of purposes. During pregnancy, for example, exosomes inhibit the production of certain T-cells thereby protecting the fetus. In the case of certain bacterial infections, exosomes derived from infected cells express antigenic fragments of the bacterium to stimulate the immune system against the pathogen. It has been postulated that cancers use the immunomodulatory properties of exosomes in order to evade the immune system. The extracellular vesicles may be specific for a disease, disorder, or condition.

In some embodiments, the exosomes are cancer cell-derived exosomes. The cancer cells may be from any cancer, including, but not limited to, breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer, or skin cancer.

Vesicle uptake can be measured by any method or assay which facilitates visualization or quantitative, semi-quantitative, or qualitative determination of uptake levels in the one or more cell types. Vesicle uptake can comprise microscopy or other imaging methods, spectroscopy, flow cytometry, and combinations thereof.

In some embodiments, the methods further comprise labeling the extracellular vesicles with a detectable marker. The detectable marker may be utilized to measure uptake of the labeled extracellular vesicles into the one or more cell types.

In some embodiments, the sample is processed to label the extracellular vesicles with a detectable marker prior to the incubation. In some embodiments, the extracellular vesicles are labeled with a detectable marker following the incubation with the cells.

The detectable marker may be a directly detectable signal generating marker or an indirectly detectable signal generating marker. Examples of detectable markers include fluorophores (e.g., a fluorescent dye, a fluorescent protein), radioactive isotopes (e.g., ³²P, and ³H), bioluminescent compounds, phosphorescent species, a light scattering or diffracting particle, an enzyme or enzyme substrate, and chromophores. In some embodiments, the detectable marker is a fluorophore.

The detectable marker may be bound or linked to an extracellular vesicle binding agent (e.g., protein, peptide, nucleic acid). In some embodiments, the extracellular vesicle binding agent comprises a tetraspanin protein (e.g., CD63, CD8, CD91), annexin V, or an antibody (e.g., EGFR, EpCAM). In some embodiments, the detectable marker (e.g., fluorescent protein) is cloned to an vesicle specific binding agent (like CD63, CD9, CD81) in reporter vectors transfected into the one or more cell types.

The methods may further comprise analyzing or conducting a biological assay with the sample. In some embodiments, the methods may further comprise measuring one or more biomarkers in the samples. In some embodiments, measuring one or more biomarkers comprises measuring one or more cancer biomarkers. In some embodiments, measuring one or more biomarkers comprises measuring or analyzing circulating tumor DNA. In some embodiments, the analysis further comprises quantifying the extracellular vesicles or exosomes in the sample. In some embodiments, the analysis further comprises isolating and analyzing the extracellular vesicles or exosomes for the presence or amount of a biomarker (e.g., DNA or RNA or protein or any combination thereof) or antigen present.

The methods disclosed herein may be used for early diagnosis and screening of diseases and disorders or to aid in the diagnosis and screening of diseases and disorders, for example with other sample analysis. In some embodiments, the methods may further comprise diagnosing or prognosing a disease or disorder in a subject. In some embodiments, the disease or disorder is cancer. In some embodiments, the disease or disorder is a neurodegenerative disease.

The methods may also be used for monitoring the effect of a medical procedure, treatment, or intervention. In some embodiments, the methods may further comprise determining the success of the medical procedure, treatment, or intervention.

In some embodiments, the methods further comprise treating the subject, based on the information obtained. For example, the subject may be administered one or more therapeutic agents (e.g., chemotherapeutic agents, levodopa, corticosteroids) or radiation or may undergo a surgery or other medical procedure (e.g., deep brain stimulation).

### 3. KITS

Also within the scope of the present disclosure are kits that include the disclosed devices or one or more components necessary for making or using the disclosed devices or the disclose systems, or for carrying out the disclosed methods. For example, in some embodiments, the kits include any or all of: organ- or tissue-specific cells, a solid surface or microfluidic device, any components necessary for the functionalization of the cells to the solid surface, detectable markers, and buffers. The kits may further comprise a sample comprising extracellular vesicles (e.g., exosomes).

In some embodiments, the kits may further contain materials for procuring or processing the sample.

Individual member components of the kits may be physically packaged together or separately. The components of the kits may be provided in bulk packages (e.g., multi-use packages) or single-use packages. The kits provided herein are in suitable packaging. Suitable packaging includes, but is not limited to, vials, bottles, jars, flexible packaging, and the like.

The kits can also comprise instructions for using the components of the kit. The instructions are relevant materials or methodologies pertaining to the kits. The materials may include any combination of the following: background information, list of components and their availability information (purchase information, etc.), brief or detailed protocols for using the compositions, troubleshooting, references, technical support, and any other related documents. Instructions can be supplied with the kits or as a separate member component, either as a paper form or an electronic form which may be supplied on computer readable memory device or downloaded from an internet website, or as recorded presentation.

It is understood that the disclosed systems or kits can be employed in connection with the disclosed methods.

### 4. EXAMPLES

### Materials and Methods

^{Cell}ExoChip device design and fabrication The top layer and bottom masking layer of ^{Cell}ExoChip was fabricated by standard soft lithography including mold fabrication and PDMS molding. By patterning SU8-2050 photoresist on a silicon wafer, the top and bottom masking layer molds were prepared. The top chamber layer was fabricated by pouring PDMS and PDMS curing agent mix (1:10) (Dow Corning, US) onto the silicon mold after degassing of PDMS mixture in vacuum for 10 minutes. The thin masking layer was prepared using a PDMS mixture spun on the silicon mold at 1,000 rpm for 30 seconds and followed by an incubation at 70° C. for 2 h. The top and bottom layers were cut, punched, and placed for processing of samples.

^{Cell}ExoChip device functionalization and cell biotinylation For the surface modification on the device, standard avidin-biotin chemistry was used with optimization. To elaborate, after plasma bonding between PDMS layer and slide glass, a silane solution (3 ml ethanol+120 μl silane) was injected three times and incubated 20 minutes after each injection. The devices were then injected and flushed out with ethanol as a washing step. Next, the devices were injected with a GMBS mixture (2 ml ethanol+6 μl GMBS) two times and incubated 15 minutes after each injection. Again, the devices were flushed out with ethanol. Following the second washing step, the devices were injected with avidin (1 ml of filtered PBS+100 μl of NeutrAvidin), placed in a Petri dish sealed with parafilm along with wet paper napkins, and incubated overnight in a standard refrigerator. After 1-10 days, the devices were defrosted and washed out with filtered PBS.

Biotinylation of cells Cancer cells were biotinylated for on-chip immobilization on following the EZ-LINK protocol with optimization. E-Z link biotin powder (EZ-Link Sulfo-NHS-LC-Biotin, ThermoScientific, United States) was utilized for cancer cell biotinylation during these experiments. A 10× of 300 μM biotin solution was prepared by dissolving 1.6 mg of E-Z link biotin in 10 ml of filtered PBS. This 10× solution was then diluted to 1× by incorporating 100 μl of the 10× solution with an additional 900 μl of filtered PBS. 100 μl of 1× 300 μM biotin solution was then added to 50 μl of purified cells, followed by a 1-hour incubation. Upon incubation, cells were centrifuged at 1,000 rpm for 10 mins three times to get rid of excess biotinylation solution.

Field emission scanning electron microscopy (FE-SEM) analysis In preparation for SEM imaging, the EV-containing device was treated with 2% glutaraldehyde to retain the morphology, for one hour while on ice. After being rinsed with PBS, the samples were subjected to bath concentrations of ethanol (50%, 70%, 90%, 95%, and 100%) for 10 minutes each (two times for 100%) to dehydrate the samples. Afterwards, hexamethyldislazane (HMDS) (Emsdiasum, United Sates) was used to dry the samples preceding an overnight air dry in the hood. The dehydrated samples were mounted on aluminum stubs using both carbon tape and glue and sputter-coated with gold particles to form a conductive layer. The TESCAN RISE scanning electron microscope (SEM) at the Michigan Center for Materials Characterization (MC2) at the University of Michigan was utilized for the surface imagining.

Field emission scanning electron microscopy (FE-SEM) analysis Staining of extracellular vesicles was completed using lipophilic dyes such as DiO and PKH. DiO staining was conducted for quantitative analysis of the exosome uptake by parent cells. In order to make DiO stained exosomes, 1 μl of DiO staining dye (ThermoFisher, USA) was thoroughly mixed with 300 μl of stock solution of each exosome. After 20 minutes of incubation, ultracentrifugation was performed to remove excess dye. After another ultracentrifugation for exosome purification, the precipitated pellet was suspended with PBS for downstream use.

EV uptake analysis using fluorescence microscope To compare the quantity of DiO stained exosomes taken up by parent cells, the average fluorescent intensity was calculated using Nikon's NIS Elements software. The average fluorescent intensities were then normalized by cell number. The standard deviation was calculated using the variation in average intensities across each replicate. The cells were also stained by nucleus dye, Hoechst. EV uptake was evaluated by DiO intensity in the DAPI positive cell regions. EVs can stained, pre- or post-incubation with cells by anti-tetraspanin proteins (CD63, CD8, CD91), specific cancer EV antibodies such as anti-EGFR, EpCAM, etc or annexin V (against phosphatidylserine on exosomes).

EV secretion from cells after EV introduction and nanoparticle tracking analysis To evaluate the on-chip immobilized cells' exosome secretion in response to foreign exosome applications in the chip, post-incubation solution was collected in the chip and centrifuged at 1,000 rpm for 10 mins to remove potential cancer cells or apoptotic bodies larger than 500 μm. The effluent solution was further evaluated using nanoparticle tracking analysis to evaluate the size distribution of the solution and concentration. 30 μl of the resultant was used and a laser module was mounted inside the main instrument housing. Based on the Brownian motion of nanoparticles, this equipment visualizes the scattered lights from the particles of interest. This movement was monitored through a video sequence for 20 seconds in triplicate. All data acquisition and processing were performed using NanoSight NS300 control software. (Screen gain: 7, camera level: 13, detection threshold: 5)

### Example 1

### ^{Cell}ExoChip

Through the organotropism properties of cells and their secreted extracellular vesicles (EVs), their characteristic interactions can provide insight into profiling the exosomes which would allow the ability to screen organ specific EVs from biological samples. Initial results from cell-EV interaction show cellular feedback from the stimulation from different organ derived EVs. For instance, a lung cancer cell would preferentially uptake lung cancer derived exosomes in comparison to bladder cancer derived exosomes. Additionally, the cellular response to the presence of different EVs can result in the secretion of that cell's own exosomes. Using the ^{Cell}ExoChip device, these unique interactions between parent and foreign derived exosomes area able to be studied to provide a means of exosome profiling in heterogenous biological samples.

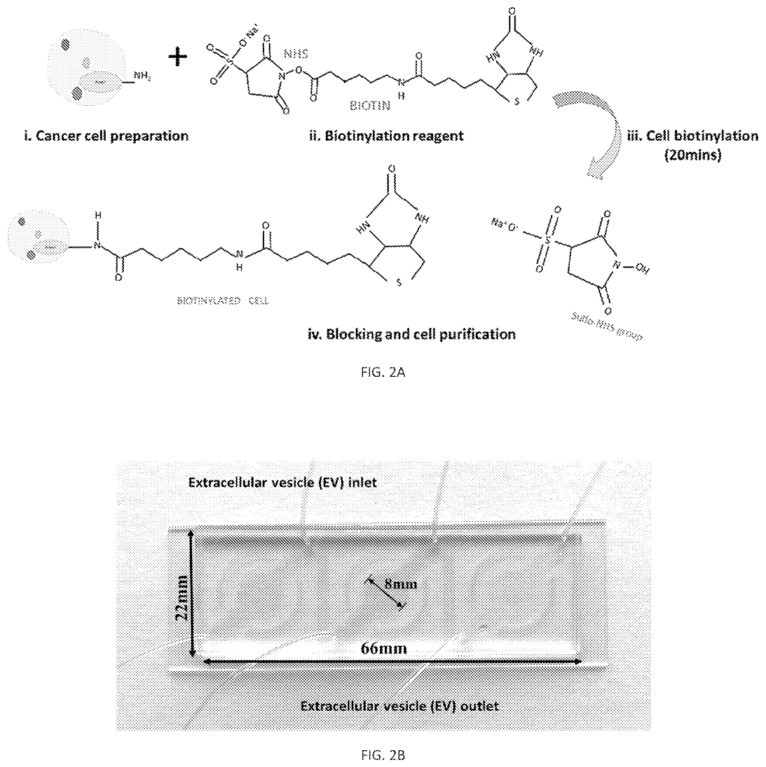

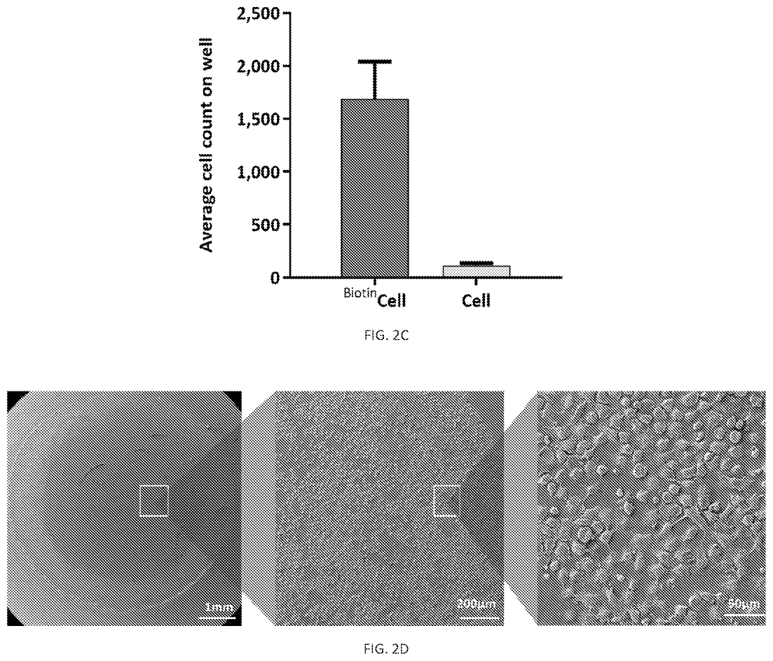

The ^{Cell}ExoChip design utilized a biotinylation reagent which specifically binds with cells and the NeutraAvidin conjugated PDMS microfluidic device (FIG. 2A). After cell conjugation on the device chamber surface, EVs from varying progenitor cells can be introduced and their interactions observed (FIG. 2B). With the use of microscopic imaging and (CellCountProgram), cell conjugation on the device was determined. As shown in FIG. 2C, the biotinylated cells had a higher conjugated cell count than the untreated cells. The non-biotinylated cells which were attached to the device were likely a result of random, non-specific binding to the device. Scanning Electron Microscope (SEM) Imaging of the ^{Cell}ExoChip device provided visual confirmation of cellular conjugation (FIG. 5D).

### Example 2

### Cell Viability and Cell-Exosome Uptake

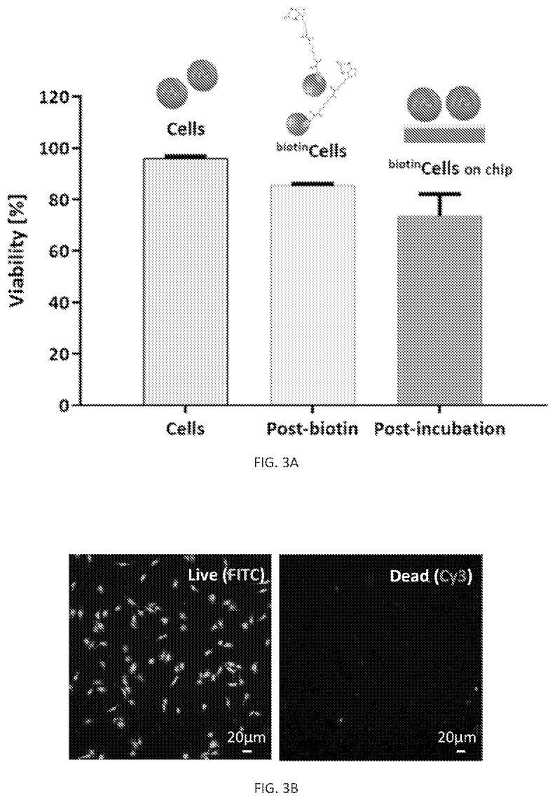

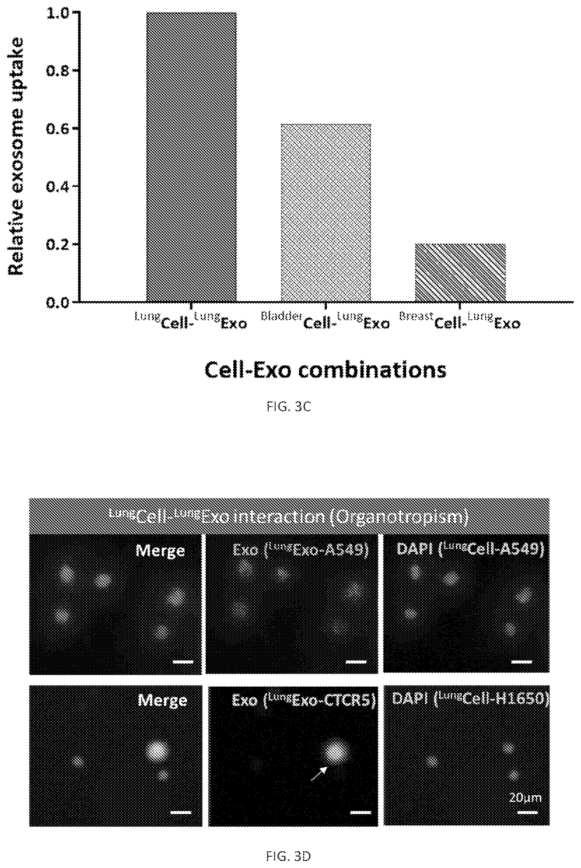

After successful conjugation, there was concern for the cell viability following biotinylation. Viability measurements were taken before and after biotinylation as well as after a 12-hour incubation on the chip (FIG. 3A). Even after the incubation, biotinylation and chip incubation had minimal effect on the cell viability, thereby allowing for further experimentation. Fluorescent microcopy showed only a small number of cells were killed because of the protein conjugation (FIG. 3B). Initial experimentation of lung cancer cells conjugated on ^{Cell}ExoChip showed that cell uptake was greatest for the lung cancer derived exosomes when compared with EVs from foreign progenitor cells (FIG. 3C). Additionally, fluorescent microscopy showed the uptake of lung cancer derived exosomes by different lung cancer cell types further demonstrating the organotrophic relationship between cells and exosomes (FIG. 3D).

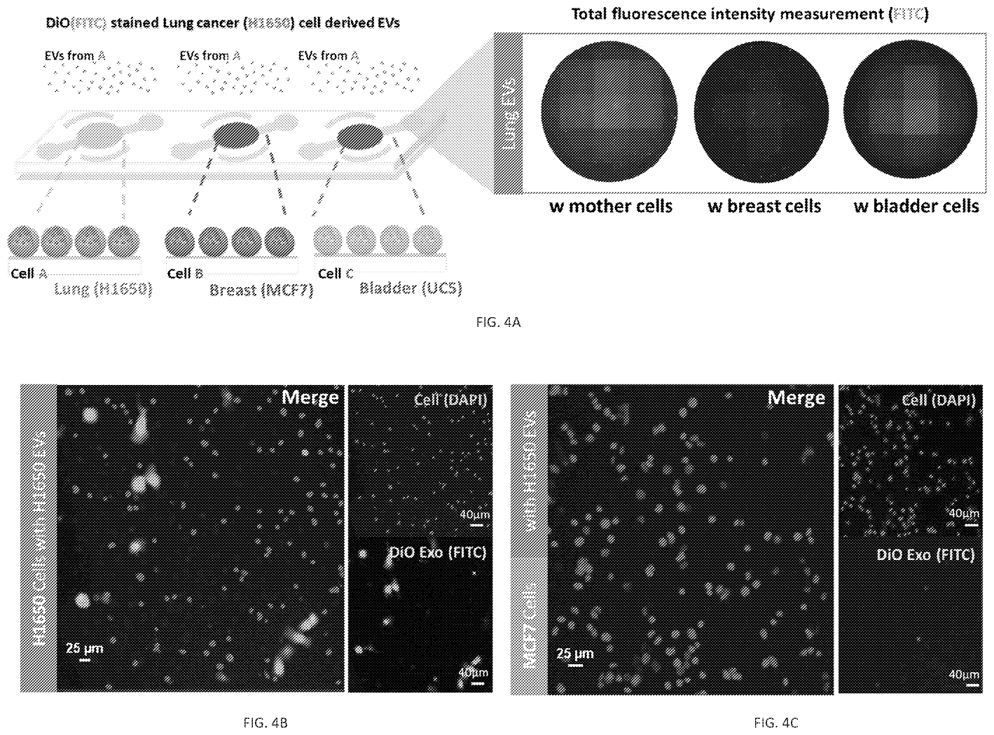

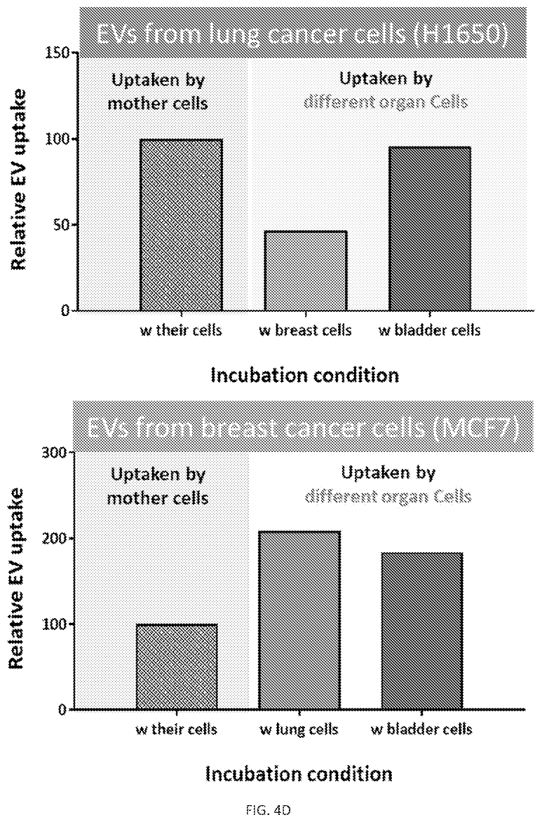

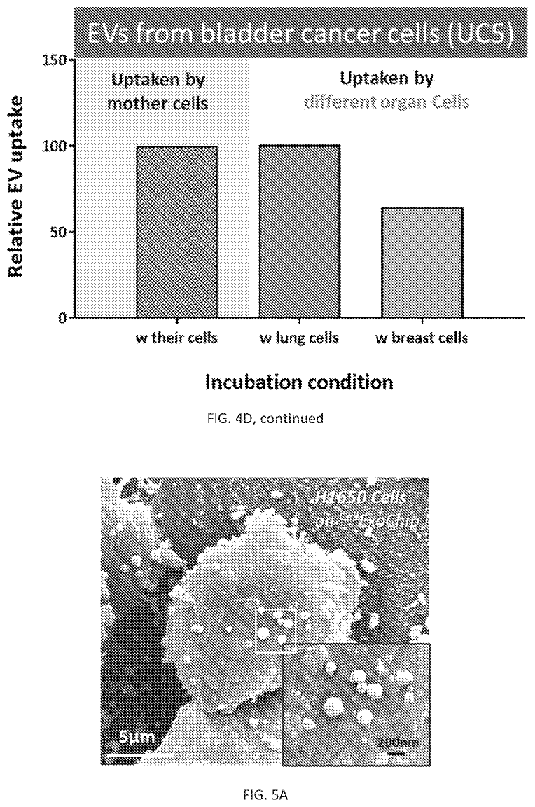

Exosomes from the lung cancer mother cell, H1650, were added to three cell types: its progenitor, bladder cancer, and breast cancer cells. Fluorescent microscopy showed the relative uptake of the EVs on the ^{Cell}ExoChip device with the exosomes preferentially being up taken by the mother cells followed by bladder then breast cancer cells (FIG. 4A). Additionally, using specific fluorescent dyeing, uptake of the lung cancer EVs into the different cell types was also monitored (FIGS. 4B-4C). In FIG. 4B, exosomes (green) were captured into the lung cancer cells (blue) while very little lung cancer exosome uptake was seen into the breast cancer cells (FIG. 4C). This data further supports the characteristic, organotrophic properties that exosomes have with varying cell types. The relative captured exosomes were quantified using fluorescent intensity revealing that the cell types not only have organotrophic properties but may also have fundamental exosome capture rates regardless of the origin of the exosomes. For example, the breast cancer cells showed less exosome uptake even when subjected to EVs deriving from the same cell type. Also, the lung and bladder cells had the largest and second largest EV uptakes, respectively, regardless of the type of the progenitor cell (FIG. 4D).

In the process of subjecting the cells to exosomes from varying progenitor sources, the EVs acted as a trigger for the cells to secrete their own exosomes. The SEM image in FIG. 5A shows exosomes being absorbed and secreted on the ^{Cell}ExoChip device. Using NTA analysis, the cell secretions were measured for the three cell types—bladder, lung, breast—and resulted in the lung (H1650) cells having the highest secretion and bladder (UC5) cell having the lowest (FIG. 5B). Additionally, the behavior of the cells was altered in the presence of their own EVs. The secretions of the three cell types were measured and each of them secreted significantly less exosomes after being subjected to EVs deriving of their cell type (FIG. 5C). It is possible that this effect is the result of the cells ability to sense and maintain a desired concentration of their own exosomes in their environment. The cell secretions were measured for the three cell types when subjected to both its own and foreign exosomes (FIG. 5D). Overall, the lung cancer derived exosomes triggered the highest cell EV secretion for differing cells followed by bladder then breast. When subjected to their own exosomes, their secretions continued to show their minimum values.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A method comprising:
incubating a sample comprising extracellular vesicles with one or more cell types;
measuring uptake of the extracellular vesicles in each of the one or more cell types; and
determining cellular origin of the extracellular vesicles.

2. The method of claim 1, wherein the cellular origin of the extracellular vesicles in the sample is that origin of the one or more cell types having increased uptake levels of the extracellular vesicles in the sample when compared to control uptake levels of the one or more cell types for extracellular vesicles of a different origin.

3. The method of claim 1, wherein the one or more cell types comprises at least one organ- or tissue-specific cell type.

4-5. (canceled)

6. The method of claim 1, wherein measuring the uptake comprises comparing uptake between each of the two or more cell types and calculating a relative uptake value for each of the two or more cell types.

7. The method of claim 6, wherein the origin of extracellular vesicles is determined as the cell type having highest relative uptake value from the two or more cell types.

8. The method of claim 1, wherein the one or more cell types comprises cancer cell types.

9. The method of claim 1, wherein the one or more cell types are immobilized in discrete locations on a solid support.

10. (canceled)

11. The method of claim 9, wherein the solid support comprises a microfluidic device.

12. (canceled)

13. The method of claim 1, wherein the extracellular vesicles comprise cancer cell-derived exosomes.

14. The method of claim 1, further comprising labeling the extracellular vesicles with a detectable marker prior to or following incubating the sample with one or more cell types.

15-16. (canceled)

17. The method of claim 14, wherein the detectable marker comprises a fluorophore, wherein measuring the uptake comprises detecting fluorescence in the one or more cell types.

18. The method of claim 14, wherein the detectable marker comprises an extracellular vesicle binding agent.

19. The method of claim 18, wherein the extracellular vesicle binding agent comprises a tetraspanin protein, annexin V, or an antibody.

20-21. (canceled)

22. The method of claim 1, wherein the method further comprises measuring extracellular vesicle secretion from the one or more cell types, wherein determining the origin of extracellular vesicles is further determined by the cell type having a minimal amount of vesicle secretion.

23. (canceled)

24. The method of claim 1, wherein the method further comprises measuring one or more biomarkers in the sample.

25. The method of claim 24, wherein the one or more biomarkers comprises a cancer biomarker.

26. (canceled)

27. The method of claim 1 any of claims 1-26, wherein the sample is a biological sample obtained from a subject.

28. (canceled)

29. The method of claim 27, wherein the subject has or is suspected of having a disease or disorder or had or is having a medical procedure, treatment, or intervention.

30-31. (canceled)

32. The method of claim 1, further comprising diagnosing or prognosing a disease or disorder in the subject.

33. A system comprising
a microfluidic device comprising one or more incubation chambers, each chamber comprising cells of a single type tethered to a surface of the chamber; and
a sample comprising extracellular vesicles.

34-36. (canceled)
